(19) United States
(12) Patent Application Publication
(10) Pub. No.: US 2005/0251976 A1
(43) Pub. Date: Nov. 17, 2005

(54) Processing device and method of operating the device for processing a coated or uncoated fibrous web

(76) Inventors: Juha Lipponen, Jarvenpaa (FI); Vilho Nissinen, Numminen (FI); Pekka Koivukunnas, Jarvenpaa (FI); Mika Viljanmaa, Helsinki (FI); Henri Vaittinen, Jarvenpaa (FI); Reijo Pietikainen, Jarvenpaa (FI); Kari Hasanen, Kerava (FI); Risto Sutti, Tampere (FI)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/502,864

(22) PCT Filed: Jan. 29, 2003
(86) PCT No.: PCT/FI03/00068

§ 371 Date: May 24, 2005

(30) Foreign Application Priority Data
Jan. 29, 2002 (FI) 20020159

(51) Int. Cl. B65D 1/00; A61F 2/00; B65H 1/00; A44B 1/00

(52) U.S. Cl. 28/100

(57) ABSTRACT

A processing device and a method of applying the same for processing a coated or uncoated fibrous web are provided. The device comprises a belt configured to extend around a guiding element, at least one counter-element being disposed outside said belt to establish a contact area with the belt, such that the belt and the counter-element establish therebetween a web processing zone for passing a web to be processed therethrough. The processing zone length is defined by the disposition/adjustment of the belt's guiding element and/or by the design of the counter-elements. The contact pressure applied to a web in the processing zone is configured to be within the range of between about 0.01 MPa and about 70 MPa.

### BACKGROUND OF THE INVENTION

1. Field of the Invention

The present invention relates to a processing device and a method of operating the device for processing a coated or uncoated fibrous web, such as e.g. paper, board or tissue, comprising a belt adapted to extend around at least one guiding element, at least one counter-element being disposed outside said belt to provide a contact area with the belt, such that the belt and the counter-element establish therebetween a web processing zone for passing a web to be processed therethrough. In the concept of this application, the term ‘web processing’ refers to a variety of measures associated with the treatment of a fibrous web produced in a paper/board machine, such as pressing, drying, calendering, coating, sizing. The processing device may also be a finishing device for a fibrous web, such as e.g. a separate coating device or printing device.

2. Description of Related Art

Various belt calender solutions have been disclosed previously e.g. in Finnish patent 95061, as well as in Finnish patent applications FI 971343 and FI 20001025. However, these belt calenders are only suitable for calendering certain grades of paper or board.

Paper and board are available in a wide variety of types and can be divided according to basis weight in two grades: papers with a single ply and a basis weight of 25-300 g/m² and boards manufactured in multi-ply technology and having a basis weight of 150-600 g/m². It should be noted that the borderline between paper and board is flexible since board grades with lightest basis weights are lighter than the heaviest paper grades. Generally speaking, paper is used for printing and board for packaging.

The subsequent descriptions are examples of values presently applied for fibrous webs, and there may be considerable fluctuations from the disclosed values. The descriptions are mainly based on the source publication Papermaking Science and Technology, section Papermaking Part 3, edited by Jokio, M., published by Fapet Oy, Jyväiskylä 1999, 361 pages.

Mechanical-pulp based, i.e. wood-containing printing papers include newsprint, uncoated magazine and coated magazine paper.

Newsprint is composed either completely of mechanical pulp or may contain some bleached softwood pulp (0-15%) and/or recycled fiber to replace some of the mechanical pulp. General values for newsprint can probably be regarded as follows: basis weight 40-48.8 g/m², ash content (SCAN-P 5:63) 0-20%, PPS s10 roughness (SCAN-P 76-95) 3.0-4.5 μm, Bendtsen roughness (SCAN-P 21:67) 100-200 ml/min, density 600-750 kg/m³, brightness (ISO 2470:1999) 57-63%, and opacity (ISO 2470:1998) 90-96%.

Uncoated magazine paper (SC=supercalendered) usually contains mechanical pulp to 50-70%, bleached softwood pulp to 10-25%, and fillers to 15-30%. Typical values for calendered SC paper (containing e.g. SC-C, SC-B, and SC-A/A+) include basis weight 40-60 g/m², ash content (SCAN-P 5:63) 0-35%, Hunter gloss (ISO/DIS 8254/1) <20-50%, PPS s10 roughness (SCAN-P 76:95) 1.0-2.5 μm, density 700-1250 kg/m³, brightness (ISO 2470:1999) 62-70%, and opacity (ISO 2470:1998) 90-95%.

Coated magazine paper (LWC=light weight coated) contains mechanical pulp to 40-60%, bleached softwood pulp to 25-40%, and fillers and coaters to 20-35%. General values for LWC paper can be regarded as follows: basis weight 40-70 g/m², Hunter gloss 50-65%, PPS s10 roughness 0.8-1.5 μm (offset) and 0.6-1.0 μm (roto), density 1100-1250 kg/m³, brightness 70-75%, and opacity 89-94%.

General values for MFC paper (machine finished coated) can be regarded as follows: basis weight 50-70 g/m², Hunter gloss 25-70%, PPS s10 roughness 2.2-2.8 μm, density 900-950 kg/m³, brightness 70-75%, and opacity 91-95%.

General values for FCO paper (film coated offset) can be regarded as follows: basis weight 40-70 g/m², Hunter gloss 45-55%, PPS s10 roughness 1.5-2.0 μm, density 1000-1050 kg/m³, brightness 70-75%, and opacity 91-95%.

General values for MWC paper (medium weight coated) can be regarded as follows: basis weight 70-90 g/m², Hunter gloss 65-75%, PPS s10 roughness 0.6-1.0 μm, density 1150-1250 kg/m³, brightness 70-75%, and opacity 89-94%.

HWC (heavy weight coated) has a basis weight of 100-135 g/m² and can be coated even more than twice.

Chemical-pulp produced, woodfree printing papers or fine papers include uncoated—and coated—chemical-pulp based printing papers, in which the portion of mechanical pulp is less than 10%.

Uncoated chemical-pulp based printing papers (WFU) contain bleached birchwood pulp to 55-80%, bleached softwood pulp to 0-30%, and fillers to 10-30%. The values with WFU are highly unstable: basis weight 50-90 g/m² (up to 240 g/m²), Bendtsen-roughness 250-400 ml/min, brightness 86-92%, and opacity 83-98%.

In coated chemical-pulp based printing papers (WFC), the amounts of coating vary widely in accordance with requirements and intended application. The following are typical values for once- and twice-coated, chemical-pulp based printing paper: once-coated basis weight 90 g/m², Hunter gloss 65-80%, PPS s10 roughness 0.75-2.2 μm, brightness 80-88%, and opacity 91-94%, and twice-coated basis weight 130 g/m², Hunter gloss 70-80%, PPS s10 roughness 0.65-0.95 μm, brightness 83-90%, and opacity 95-97%.

Release papers have a basis weight within the range of 25-150 g/m².

Other papers include e.g. sackkraft papers, tissues, and wallpaper bases.

Board making uses chemical pulp, mechanical pulp and/or recycled pulp. Boards can be divided e.g. in the following main groups according to applications thereof.

Corrugated board, comprising a liner and a fluting.

Boxboards, used for making boxes and cases. Boxboards include e.g. liquid packaging boards (FBB=folding boxboard, LPB=liquid packaging board, WLC=white-lined chipboard, SBS=solid bleached sulphite, SUS=solid unbleached sulphite).

Graphic boards, used for making e.g. cards, files, folders, cases, covers, etc.

One board grade comprises wallpaper bases.

### SUMMARY OF THE INVENTION

As can be appreciated from the above, there is a wide range of paper and board grades, and a multitude of various machines are used for making the same. Hence, it is an object of the present invention to provide a processing device and a method of operating the same, allowing the use of a highly extensive pressure range and application or action time (heat transfer time and/or processing time) in a processing zone, the same device being applicable for processing a wide variety of coated and uncoated printing papers, boards and other papers, and being applicable e.g. as a preliminary calender upstream of coating, a finishing calender downstream of a paper machine or coating, a breaker stack, a wet stack calender, or as a dryer, a coater, a sizer, a printer and/or a press. The inventive device is conceivable as a replacement e.g. for a soft calender, a multi-nip calender, a machine calender, a shoe calender, or a Yankee cylinder.

In order to fulfill the objects of the invention, a device of the invention is characterized in that the processing zone length is defined by means of the disposition of the belt's guiding element and/or by means of the design of the counter-elements, and that the contact pressure applied to a web in the processing zone is adapted to be adjustable within the range of about 0.01 MPa to about 200 MPa.

On the other hand, a method of the invention for processing a coated or uncoated fibrous web with a processing device is characterized in that the method comprises defining the processing zone length by means of the disposition of the belt's guiding element and/or by means of the design of the counter-element, and that the method comprises adjusting a contact pressure existing in the processing zone to lie within the range of about 0.01 MPa to about 200 MPa.

Contact pressure refers to the sum of pressure effects applied to a web within a processing zone between a belt and a counter-element, which are caused by a tension of the belt and/or by a compression force applied by possible intra-belt press elements. The pressure adjustment of a contact pressure to a certain pressure value or pressure range is effected by choosing a suitable belt material, which allows the use of a desired tightness or tension, and, if necessary, suitable press elements capable of increasing pressure in a localized manner over what is achieved by the belt alone. It should be noted that, depending on an assembly made up by belt and counter-elements as well as possible press elements, it is possible to cover either a part of the contact pressure adjustment range, the transition to another pressure value or pressure range being effected by replacing, if necessary, some of the elements included in the assembly, or to cover, with a suitable assembly, the entire contact pressure adjustment range, which can be e.g. from about 0.01 MPa to about 70 MPa or even from about 0.01 MPa to about 200 MPa. For example, the compression achieved by belt tension alone is remarkably low when compared to the compression accomplished with press elements, whereby, in the solutions implemented without press elements, the adjustment range lies closer to a lower limit, e.g. within the range of about 0.01 MPa to about 5 MPa. When using press elements, the adjustment range can be e.g. from about 5 MPa to about 70 MPa, preferably from about 7 MPa to about 50 MPa or e.g. from about 70 MPa to about 200 MPa.

The inventive device comprises preferably a calender, a coater, a sizer, a printer, a dryer, a press, and/or a web cooler. According to the invention, a number of the above devices can be set successively in a common fibrous web production line, the sequence being for example a press device, a drying device, a calender, web cooling.

In addition, the inventive device preferably comprises doctor or other elements provided downstream of the processing zone for cleaning the belt of a metal belt calender over its side facing the paper web. This way, the belt surface can be cleared of lumps of pitch and dirt deposited thereon from the paper web in a nip. Moreover, the inventive device preferably comprises elements for cooling the heated belt of a metal belt calender along its edges. Cooling can be effected in a per se known manner, for example by means of water or air injection. Cooling is particularly preferred in the case of running a paper or board web which is narrower than the belt. By cooling the edges, it is possible to eliminate a high temperature difference otherwise present along the edge of a web and to relieve the metal belt of stress conditions resulting therefrom and eventually causing fatigue failures.

One object of the invention is to provide a method for quickly switching a grade of coated or uncoated paper, board or tissue to be produced in a paper/board machine from one grade to another. The method is implemented by means of a processing device, comprising a belt adapted to extend around guiding elements, at least one counter-element being disposed outside said belt to provide a contact area with the belt, such that the belt and the counter-element establish therebetween a processing zone for passing a web to be processed therethrough. The method is characterized in that the method comprises providing the belt with heating means and/or cooling means for quickly changing the belt temperature, and that the adjustment of a temperature applied to the web is essentially performed by only adjusting the belt temperature.

An object of the invention is a profiling nip established in an embodiment implemented as a processing device provided with a metal belt, and an arrangement for the grade-specific adjustment of a nip length.

A device according to this additional aspect of the invention is characterized in that outside the processing zone is arranged a deflection-compensated nip roll establishing a profiling nip with a roll, the web being adapted to travel through said profiling nip, that the processing zone length is defined by means of the disposition of the belt's guiding element and/or by means of the dimensioning of the roll, and that a contact pressure applied to the web in the processing zone is adapted to be adjustable within the range of about 0.01 MPa to about 200 MPa.

The essential concept in this aspect of the invention is that, in addition to a processing zone (i.e. a long nip), there is provided at least one profiling nip. The assembly is roughly consistent with a soft calender. The inventive solution is readily capable of providing e.g. desired surface qualities. Furthermore, the solution is structurally simple. It makes it possible that, if desirable, the components essential in terms of proper operation of the device be positioned entirely outside a belt loop.

The present invention relates to a device for drying a fibrous web, said device comprising a dense, air impermeable metal belt, which forms a surface P1 and is adapted to extend around at least one guiding element, at least one counter-element, which forms a surface P2, being disposed outside said belt to provide a contact area with the belt, the surfaces P1 and P2 establishing therebetween a fibrous web drying zone for passing the to-be-dried fibrous web through said drying zone, wherein one of the surfaces P1 and P2 is heated and the other is cooled, at least one porous wire being further adapted to travel between the surface P1 and the surface P2, such that the fibrous web is in contact with the heated surface and the wire is in contact with the cooled surface, the steam which escapes from the fibrous web migrating through the wire to the cooled surface and condensing thereon.

The CondeBelt-method, based on condensation drying, for drying paper and board is known e.g. from publications FI 97485 B1 and FI 99272 B1. In the solutions of these publications, a fibrous web to be dried by means of condensation drying is passed or guided, together with a porous wire, in between two dense, highly heat conductive surfaces, for example metal belts. The belt, which is in contact with the fibrous web, is heated and, thus, there is a transfer of heat to the web to vaporize water therein. At the same time, the other belt, lying against the wire, is cooled, the vaporized water, which has migrated through the wire, condensing on the surface of the metal belt.

In this context, it is particularly notable that the CondeBelt-process requires a sufficiently long dwell time in order to function properly. For example, the 1-5 ms dwell time of conventional nips is too short. Thus, practicing a solution like the CondeBelt-method creates a need to employ a sufficiently long contact area for the effective drying of a fibrous web.

The prior art embodiments of the CondeBelt-process make use of the condensation of pressurized steam to provide heating for the hot side wire and, on the other, pressurized cooling water for cooling the cold side, as described in the cited publication FI 97485 B1. On the other hand, in the solution of the cited publication FI 99272 B1, the hot side heating is effected by means of pressurized hot water. One crucial function of the pressurization is to sustain the equilibrium of forces existing on opposite sides. As a downside, however, this type of solution requires awkward, expensive and high-maintenance insulations and pressurized chambers. Regarding its overall structure, the CondeBelt-type of implementation has also the drawback that the length of a drying zone cannot be manipulated any longer, nor can it be readjusted after installation.

On the other hand, the preamble of this application discloses a processing device, applicable to processing a paper and board web and provided with a metal belt, and a technical principle for the implementation thereof. It comprises using a belt and a counter-element disposed outside the belt to establish a compression contact substantially longer than in presently available calenders, the length and compression pressure thereof being variable and adjustable in a versatile manner.

A target of the invention is to provide a solution which combines the benefits gained by a metal-belt provided processing device of the invention and those offered by the CondeBelt-process. This enables, on the one hand, making use of the beneficial aspects of the CondeBelt-process for the effective and high-speed drying of a fibrous web as well as, on the other hand, the advantages of a metal-belt provided processing device in terms of a wide-range adjustability for the length of a fibrous web processing zone and the fibrous web processing time, and also for other process parameters.

In order to implement this additional aspect of the invention, a device of the invention is characterized in that the belt extends about a counter-element in compliance with its surface, that the drying zone length is adapted to be adjustable by means of the disposition of the belt's guiding element/elements and/or by means of the design of the counter-element/elements, and that a contact pressure applied to the fibrous web is adapted to be adjustable within the range of about 0.01 MPa to about 70 MPa, such that the contact pressure applied to the fibrous web in the drying zone is higher than a steam pressure generated in the vaporization process of water contained in the fibrous web.

The inventive solution provides a device for drying a fibrous web, which enables the use of a very extensive application or action time (heat transfer time and processing time) and temperature range, as well as pressure range, in a drying zone.

One object of the invention is a processing device for compressing a coated or uncoated fibrous web, such as a web of paper and board, wood fiber board, for packing and/or dewatering the web. The processing device comprises a belt adapted to extend around at least one guiding element, at least one counter-element being disposed outside said belt to provide a contact area with the belt, such that the belt and the counter-element establish therebetween a web processing zone for passing a web to be processed therethrough.

The function of a paper machine's press section is to remove water from a wet fibrous web (dry content 15-20%) as effectively as possible, such that, upon passing over to a dryer section, the dry content would be as high as possible (in practice about 50-55%). In contemporary wet presses, a web is conveyed to a nip contact, wherein the web is compressed against an absorbent felt in such a way that water is driven under the force of pressure loads from web to felt. Commonly applied are press solutions based on a conventional roll nip or a so-called shoe nip. The press nip can be provided either with a single felt lining, whereby a felt is present on one side of the web only, or with a double felt lining, whereby water escapes into felts present on either side.

In addition to conventional wet pressing, which involves no heating of a paper web to be dried, there are also a variety of prior known hot pressing methods. Hot pressing involves heating a web, yet the web temperature does not exceed 100° C. If this temperature is exceeded, the process is called super-hot pressing or high intensity pressing. Another applied term is impulse pressing. The principle of hot pressing methods is to enhance a press drying process both by lowering the viscosity of water as a result of heating and by bringing the water to vaporization at some point in the compression process.

The principal dewatering mechanisms can be divided roughly into three main categories. Firstly, dewatering may occur as a result of the packing or compaction of the composition. Heating also enhances flowing by reducing the viscosity of water. This type of mechanism is dominating in conventional wet pressing and hot pressing. Secondly, dewatering may take place by way of the development or expansion of steam, i.e. water is displaced by steam, the term applied being so-called impulse pressing. In order to create an impulse effect, the temperature must be sufficiently high for water to vaporize at a presently applied pressure. A third mechanism comprises flashing or flash-drying. It involves a transfer of so much heat to paper that temperature exceeds 100° C., yet the water is not immediately vaporized at a presently applied pressure but, instead, the vaporization occurs suddenly only as a result of a pressure drop as the nip opens. Thus, the water escapes from a fibrous web simply by vaporization. All these phenomena are involved in hot pressing, but the impact thereof on dewatering depends on case-specific conditions.

Regarding the function of a press, it is an essential feature whether the pressing process is “compression limited” or “flow limited”. In the former case, the compression of a composition sets a limit to dewatering, and pressing can only be enhanced basically by increasing the compression pressure. In the latter case, on the other hand, the limit is set by a viscous flow resistance, and dewatering can be enhanced by extending a dwell time. In a device of the invention, introduced in the preamble, for processing a coated or uncoated fibrous web, an essential component is a belt loop or circle, which enables setting a fibrous web in a substantially longer-than-before pressing contact with a counter-element. The belt solution also provides an easier-than-before control over the length and pressure of a contact zone. Furthermore, a fibrous web can be heated effectively by means of the belt. Thus, an essential concept according to this aspect of the invention is to apply the technical benefits offered by a processing device set forth in the preamble to a pressing process, and particularly to a drying process.

A device of the invention is characterized in that the device is provided between a belt and a counter-element with at least one porous felt and/or wire for receiving the water removed from a fibrous web, that the processing zone length is defined by means of the disposition of the belt's guiding element and/or by means of the design of said at least one counter-element, and that a contact pressure applied to the web in the processing zone is adapted to be adjustable within the range of about 0.01 MPa to about 70 MPa.

Essentially, the idea is to increase the length of a pressing zone and thus to extend the application or action time of a press. This is accomplished by designing the passage of a belt loop to be modifiable by means of guiding or guide rolls, whereby the length of a press zone (“overlap angle”) can be readily adjusted. The belt loop is supported and guided by means of separate guiding rolls, the location and/or position of one or more guiding rolls being preferably adjustable.

By virtue of a longer dwell time, the inventive solution is capable of achieving a higher dewatering rate, especially in flow-limited circumstances. In addition, the opening stage, i.e. the emergence of a web from a pressing zone, can be controlled better than before for thus avoiding delamination, which may occur if the opening takes place too quickly.

The method is particularly suitable for just, a so-called press section, wherein the dewatering is primarily based on mechanical compression and a supplementary thermal effect (change of viscosity, vaporization), by using traditionally a conventional press, a shoe press, a hot press, or a an impulse drier.

In terms of press section, the notable essential benefits offered by the method include first a long and better-than-before adjustable pressing zone. The zone length, the efficiency of heat transfer, and the distribution of a compression load (pressure profile) are more easily controllable. A particular advantage in this respect is achieved in hot pressing and impulse pressing, in which the above-mentioned delamination constitutes a problem. In addition, the novel solution and selection of proper materials provide an adjustment window substantially more extensive than before for the process and make the process more effective. Moreover, the dosage of heat can be effected in a more versatile manner. For example, the induction heating of a metal belt can be implemented upstream of or within the zone.

The method is also applicable to dryer section, wherein the most significant benefits gained thereby include a more effective contact with paper compared to contemporary drying wires/cylinders (contact pressure substantially higher than what is achieved by wires), a possibility of a two-sided supply of heat (more symmetrical drying result and mechanical properties), as well as a possibility of finishing and glazing the paper surface by means of a smooth belt.

A conventional pressing process is primarily based on the compression of a web for reducing a pore volume and squeezing a corresponding amount of water out of the composition. Hence, during the course of compression, the web remains substantially impregnated with water, without hardly any gas phase existing or evolving. On the other hand, impulse pressing or drying, wherein the top surface of a web is subjected by a thermal roll to such a powerful heating that water vaporizes and the expanding steam forces water to flow out of the web, is, at least on the level of principle, based on the displacement action of vaporized water (gas). Liquid phase (water) is being blown by water vapor out of the composition to be dried.

On the other hand, in terms of controlling the operation of a press, a highly essential feature is the selection of a correct pressure level and distribution, as well as a nip dwell time. In a shoe press, the pressure level is in the order of a few MPa's, the dwell time in high-speed machines being in the order of less than 10 ms, and even otherwise commonly in the order of less than 40 ms. In roll presses, the pressure is higher but the dwell time is less than 2 ms. Generally speaking, it can be concluded that, especially in flow-limited pressing conditions (high-caliper grades), it is considerably more preferable to increase the application time of a pressing pulse than the maximum pressure. Hence, the trend is towards a longer pressing zone.

Accordingly, it is one object of the invention to provide a method, in which the drying of a fibrous web effected by means of pressing is enhanced, such that the pressure of a compressed gas present in felts and a fibrous web participates in the displacement of water contained in the fibrous web, and which is applicable to wet pressing a fibrous web in paper, board and chemical pulp machines. In order to achieve this object, a method of the invention comprises drying a web of paper/board by pressing it in a processing device, comprising a belt adapted to extend around at least one guiding element, at least one counter-element being disposed outside said belt to provide a contact area with the belt, such that the belt and the counter-element establish therebetween a web processing zone for passing a web to be processed therethrough, the method being characterized in that the processing device used in the method is provided on either side of a web (W) with a pore volume, that at least on one side of the web the pore volume is established in a compressible felt/wire, in which method the fibrous web (W) to be dried is conveyed in contact with said pore volumes through the processing zone, in which the latter are subjected to a pressing action, whereby the felt/wire compresses and at the same time the pressure of a gas present in its pores increases, causing the flow of gas against the web and thus forcing the water contained in the web towards the pore volume present on the other side of the web.

The method comprises pressing a fibrous web in a long pressing zone according to the above-described invention between two bearing surfaces together with one or more porous and compressible felts/wires. Particularly preferred is the use of a pressing zone established by a metal belt and a roll. The roll-encircling belt provides a long contact, the belt tension being capable of providing a contact pressure in the order of 0-5 MPa. In addition, an extra load can be provided by using a conventional shoe roll for establishing a more localized high-pressure area. The zone length can be as much as 5 m for a truly long dwell time of 100-500 ms, even 1000 ms. The solution according to this aspect of the invention offers benefits similar to those already mentioned, such as e.g. a long and better-than-before adjustable pressing zone, the length, heat transfer efficiency and compression load distribution (pressure profile) of which are more easily controllable and in which the adjustment window for the process can be made substantially larger and the process more effective. In addition, the dosage of heat can be effected in a more versatile manner.

Furthermore, in view of controlling delamination, it is preferred that the pressure distribution of a processing zone be controllable in the web running direction. The inventive device enables a pressure control in the web running direction in a variety of ways. For example, this can be implemented by means of the design and disposition of an extra loading element fitted inside the belt loop. On the other hand, the pressure effect can be controlled separately by means of belt loops lying inside or on top of each other, i.e. in practice by independent adjustment of belt tensions. It is also conceivable that the opening points of belt loops lying inside of each other be adapted to occur successively at suitable intervals.

The invention relates to a method for treating a coated or uncoated fibrous web with a sizing agent.

The present invention has managed to combine the benefits of a pressurized thermal treatment enhancing the sizing process of a fibrous web, as well as, on the other hand, the high-speed and wide-range adjustability of process conditions provided by a processing device as described above, which is preferably executed as a metal belt calender. Thus, the invention is implemented by applying a hot, long pressing zone. The invention serves to provide a method, which enhances the sizing process of a fibrous web, is suitable for both internal and surface sizing, and further even allows the use of a very extensive pressure range and application time (heat transfer time and/or treatment time) in a processing zone, whereby one and the same device can be used for the sizing or other chemical treatment of numerous different coated and uncoated papers and boards.

In order to achieve these objects of the invention, a method of the invention is characterized in that at least one sizing process of a fibrous web to be treated with a sizing agent comprises the use of a belt device, comprising a metal belt adapted to extend around one or more guiding elements, at least one counter-element being disposed outside said belt to provide a contact area with the belt, such that the belt and the counter-element establish therebetween a processing zone for passing a web to be processed therethrough, wherein the processing zone length is defined by means of the disposition of the belt's guiding element and/or by means of the design of the counter-elements, and wherein a contact pressure applied to a web is adjusted by a belt tension and/or by the design of press elements and/or by means of a compression force applied by the press elements to the belt to lie within the range of about 0.01 MPa to about 70 MPa.

An essential feature in the inventive solution is that the process solution is arranged to be such that, by means of said readily adjustable heating and pressing zone, a web can be provided with conditions capable of bringing about a reaction of paper and added chemicals at a fairly high resulting temperature and pressure. Naturally, paper can be supplemented either with only one or more than one sizing chemicals. Useful chemicals may include, for example, resins applicable in internal and surface sizing, synthetic sizes (AKD, ASA, etc.), and other chemicals with a beneficial effect.

Accordingly, the leading concept of the invention is to execute a fibrous web sizing process by means of a loop belt included in a metal belt calender, wherein a belt extends around a counter-element in compliance with its surface over a distance equal to a sufficiently long web processing zone of an arbitrarily adjustable length. The pressing surfaces established by means of a metal belt and a counter-element make up a sort of closed pressure chamber, wherein e.g. the size softening, conveyance, fixing reaction, and bonding of sizing agents take place faster in a web treated with sizing agents. Furthermore, since the heating is effected from the surface of paper, the paper develops major temperature and concentration variations in the early stages of heating. The beneficial result is the ability of a size (size vapor) to migrate in the perpendicular direction of paper, thus equalizing the sizing effect in the perpendicular or thickness direction. Thus, even one-sided surface sizing works across the entire paper thickness.

The contact pressure of a processing zone is controlled by means of a metal belt tension. In this respect, it is essential that the pressure applied to a fibrous web be adapted in the processing zone preferably in such a way that, on the one hand, the steam pressure generated as a result of heating the fibrous web and, on the other hand, the mechanical pressure pressing the contact areas or surfaces are in equilibrium or the latter is higher. Likewise, at the trailing edge of a processing zone, the arrangement of temperature and pressure is preferably such that no delamination or excessively powerful discharge of gases can take place.

Another notable advantage of the inventive arrangement is that, by virtue of its high-speed and easy adjustability, a metal belt calender as described above can be used for quickly switching a grade of paper by adjusting the process conditions to be optimally consistent with the web thickness and properties, as well as with the compositions and amounts of sizing agents.

The described arrangement is particularly suitable for the hydrophobic and strong sizing of paper and board, but it is also conceivable to apply the arrangement for the impregnation as well as coating of paper. In the case of dry coating, the melting and bonding of a coating material may also be relevant.

One essential observation is that the same device, in which paper is pressed with a smooth, heated contact surface, is also capable of providing a calendering and drying effect. It is particularly conceivable that the enhancing treatment of fibrous web sizing processes effected by means of a metal belt calender and the calendering of a fibrous web be executed simultaneously with one and the same device. A metal belt calender, which establishes a long zone, is especially suitable for the purpose.

### BRIEF DESCRIPTION OF THE SEVERAL VIEWS OF THE DRAWING(S)

The invention and a variety of its applications will now be described in more detail with reference to the accompanying drawings, in which:

FIG. 1 shows one exemplary embodiment for a device of the invention in a schematic side view,

FIG. 1b shows one variant for the device of FIG. 1 in a schematic side view,

FIG. 2 shows a second embodiment for a device of the invention in a schematic side view,

FIGS. 3-7 show a few alternative implementations of the invention,

FIG. 8 is a schematic side view, showing one exemplary embodiment for a device of the invention, provided with a profiling nip,

FIG. 9 is a schematic side view, showing a second exemplary embodiment for a device of the invention, provided with a profiling nip,

FIG. 10 is a schematic side view, showing one exemplary embodiment for a device of the invention, applicable for drying a fibrous web,

FIG. 11 is a schematic close-up of an area A indicated in FIG. 10, visualizing a fibrous web drying process within a drying area,

FIG. 12 is a schematic side view, showing a second exemplary embodiment for a device of the invention, applicable for drying a fibrous web,

FIG. 13 shows still another embodiment for a device of the invention, applicable for drying a fibrous web,

FIG. 14 is a schematic close-up, showing a portion of still one embodiment for a device of the invention, applicable for pressing a fibrous web,

FIG. 15 is a schematic close-up, showing a portion of a second embodiment for a device of the invention, applicable for pressing a fibrous web,

FIG. 16 shows in a schematic close-up a portion of one embodiment consistent with the device of FIG. 1, applicable for drying a fibrous web,

FIG. 17 shows in a schematic close-up a portion of a second embodiment consistent with the device of FIG. 1, applicable for drying a fibrous web,

FIG. 18 shows in a schematic close-up a portion of the device of FIG. 17, visualizing a fibrous web drying process within a pressing zone/drying area,

FIG. 19 shows in a side view a pilot machine implemented in accordance with the invention,

FIG. 20 shows the pilot machine of FIG. 18 in a plan view,

FIG. 21 shows schematically one implementation for an LWC paper production line.

### DETAILED DESCRIPTION OF THE INVENTION

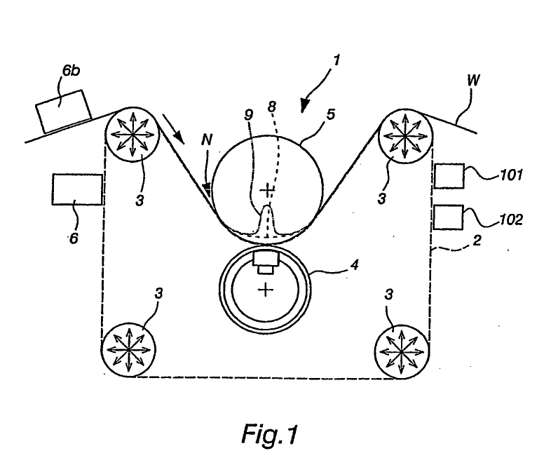

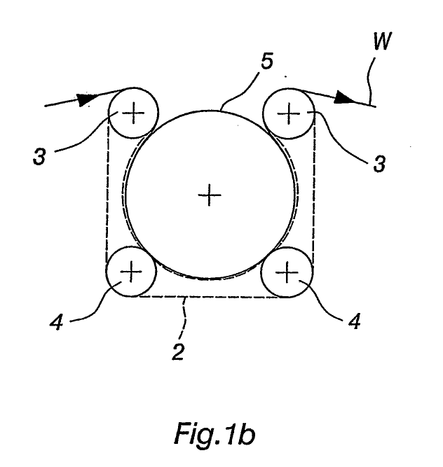

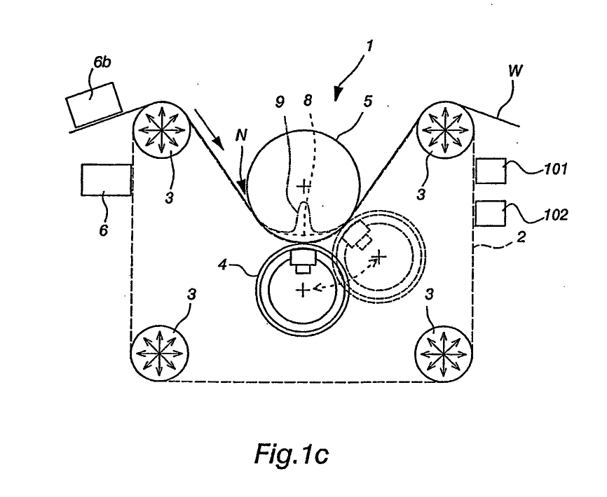

FIG. 1 illustrates one device of the invention executed as a belt calender, comprising a metal-constructed calendering belt 2, which extends around guiding rolls 3, at least some of said guiding rolls being movable for adjusting the belt 2 to a desired tension. The calendering belt 2 travels around a roll 5 disposed on the outside thereof, a calendering zone being established between the belt 2 and the roll 5. A material web W to be calendered travels through the calendering zone, being subjected to a desired pressure impulses and thermal effect as a function of time. A dash-and-dot line 9 in FIG. 1 represents the pattern of pressure curve when the calendering belt 2 is provided on the inside thereof with a nip roll 4 functioning as a press element, which compresses the belt against the roll 5 to establish a higher-pressure nip area within the calendering zone. On the other hand, a dash line 8 represents the pattern of pressure curve when the contact pressure existing in the calendering zone is created by a belt tension alone, the nip roll 4 being out of a compressing contact with the belt 2 (or when there is no nip roll 4 at all fitted inside the belt 2). The roll 5, like the nip roll 4 as well, may or may not be a deflection-compensated roll and it is selected from a group, including: an elastic surface roll, such as a polymer-covered roll, a rubber-covered roll or an elastomer surface roll, a shoe roll, a thermal roll, a filled roll, and a composite roll. Instead of the roll 4, the press element may also comprise some other profitable or fixed-profile press element, which, in addition, can be constituted by several components or elements successive in the cross machine direction. The press element 4, implemented in the form of a roll, may also be constituted by several components successive in the cross machine direction. The press element 4 may have its surface designed as continuous or discontinuous. Furthermore, the press element 4 can be designed to be movable for changing the processing zone length and/or belt tension.

In the embodiment shown in FIG. 1, the nip roll comprises a shoe roll. Reference numeral 6 represents heating elements, such as for example an induction heater, an infrared radiator, a gas burner. Heating can be based also on resistive heating. Especially in the case of a metal belt, the inventive solution can be implemented by applying elevated temperatures, for example from higher than about 100° C. to higher than about 200° C., and even up to about 400° C., depending on intended application. The elevated temperature, together with a long application time and a wide pressure control range, yields a good calendering result at both high and low speeds, e.g. at speeds of 100 m/min to 4000 m/min.

Reference numeral 6b in FIG. 1 indicates heating means with a direct effect on a paper/board web, e.g. an infrared radiator or a microwave/RF heater. Heating can be effected also resistively or with some other prior known technique.

FIG. 1b illustrates one variant for the device of FIG. 1, wherein an endless belt 2 travels around guiding rollers 3 and press rolls 4. The guiding rollers 3 are adapted to be movable for adjusting tension of the belt and the press rolls 4 are adapted to shift in a direction towards a roll 5, whereby the movement of the guiding rollers 3 enables the belt 2 to compress or force the press rolls 4 against the roll 5.

FIG. 1c shows yet another variant for the device of FIG. 1, wherein the press roll 4 is adapted to be movable.

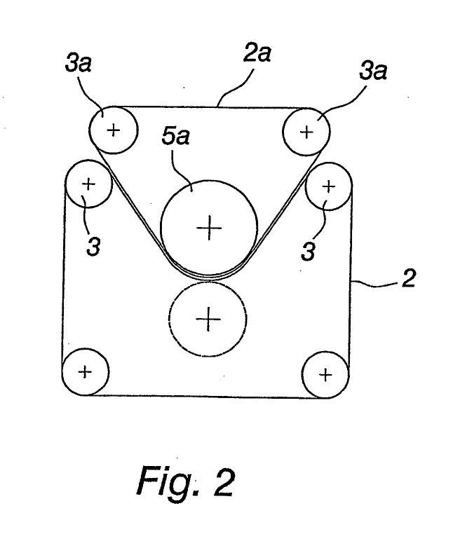

FIG. 2 illustrates an exemplary embodiment, wherein a calendering zone is established between tow calendering belts 2 and 2a, whereby a roll 5a present inside the belt 2a can be selected the same way as the above-mentioned roll 5. The belt 2 may also be provided with an inside roll for establishing a nip with the roll 5a.

The calender belt 2 used in a belt calender executed according to the invention may not comprise a metal belt as described above, but, instead, e.g. a steel-reinforced rubber belt, polymer belt or a coated metal, rubber or polymer belt. The roll 6 may also be provided with a hard or soft surface. The belt 2 and/or the roll 5 can be smooth in surface or embossed, and a contact area or surface constituted by the belt and/or the roll with a web W may move at a speed different from that of the web W. The belt coating may comprise a permanent or removable coating. The coating can be granular, liquid, solid, made of elutriated fines, and the coating may be detachable from the belt surface in a controlled fashion.

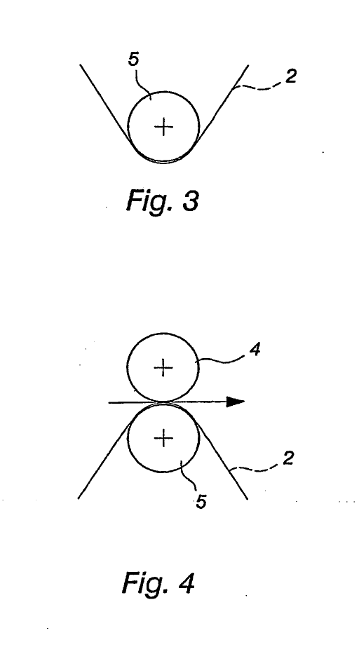

FIGS. 3-7 are schematic illustrations of a few alternative implementations for a fibrous web processing device, wherein the shape of a processing zone is designed by using various counter-elements to provide a contact area or surface with the belt and various press elements to create a desired pattern for a pressure pulse. The counter-elements and the press elements may comprise rotating or non-rotating rolls or various support bars. Said elements may also be provided with a crowning or an adjustable profiling for controlling a lateral tension and pressure effect of the belt.

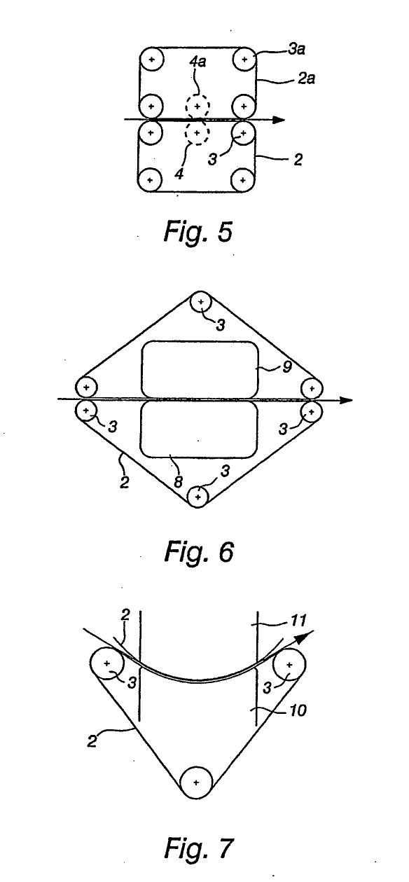

FIG. 3 illustrates a processing zone established by a belt 2 and a roll 5, wherein a pressure pulse is produced by means of the belt tension. FIG. 4 includes, in addition to the belt 2 and the roll 5, a nip roll 4 for applying an extra compression force to a presently treated web. FIG. 5 illustrates a substantially planar processing zone established between two belts 2 and 2a, which solution can also be optionally provided with intra-belt rolls 4 and/or 4a (depicted with dash lines in FIG. 5) for supporting the belt 2 or 2a over the section covering planar zone. The rolls 4 and 4a can establish a nip with each other. FIG. 6 depicts a solution, wherein two belts 2 travel under the guidance of guiding rollers 3 around two bar elements 8 and 9 constituting a substantially flat surface. A processing zone is provided between the belts 2. The intra-belt element 8 and/or 9 can be stressed or biased against the inner surface of the respective belt 2 for producing a desired pressure impulse in the processing zone. FIG. 7 shows a solution, wherein the belt 2 extends around a dish-surface bar 10 and wherein the press element comprises a convex-surface bar 11, around which runs another belt 2. A processing zone is provided between the belts 2.

A processing device of the invention is also conceivable for use in the dryer section of a paper/board machine, the belt comprising a metal belt and the counter-element, which establishes a contact area therewith, comprising a drying cylinder.

A processing device of the invention enables a supported belt passage through a processing zone and allows a controlled fluctuation of the web width within a range defined by the belt width. Web feeding is possible over a full web width and at a high web running speed.

The regulation of moisture/temperature in a presently treated web can be performed by conventional means, for example by steaming the web surface/surfaces prior to passing the web into a processing zone. The regulation of moistening/temperature can be used for a desired effect on the lateral profile of a web and the method provides a possibility for a wide-range fluctuation of web moisture.

A method of the invention provides also a possibility of cooling a metal belt or a thermal roll to a temperature of about −70° C. to +50° C.

For example, the manufacture of glossy printing paper in current technology requires an expensive multi-nip calender. A glossy surface is also producible at slow speeds, as well as by applying low pressures and low temperatures, by copying against the surface of a Yankee cylinder. However, the Yankee cylinder is limited in terms of speed and width.

The inventive belt calender allows for the use of considerable speeds, and by additionally using an elevated temperature, e.g. about 250° C., and by considering a long dwell time in a processing zone, the resulting glazing effect will be equal to what is achieved in the slower Yankee cylinder solution.

Another advantage gained by the inventive solution is a comparatively low power demand, since the transmission of energy, heat, and power to a web takes place in a single process in an enhanced manner. The heat delivered to a web or a coating layer is not able to escape from the web to ambient air but continues its temperature raising effect, thus facilitating significantly the glazing of a web surface.

The inventive device is preferably provided with means 101 downstream of a processing zone for cleaning the surface of a belt 2 facing a fibrous web W in a metal belt calender 1. This is to clear the belt surface of lumps of pitch and dirt deposited thereon from the fibrous web in the nip.

The inventive device is also preferably provided with means 102 for cooling the edges of a heated belt 2 in a metal belt calender 1. Cooling can be effected, for example, by means of water or gas injection. Cooling is particularly beneficial when running a paper or board web which is narrower than the belt. By cooling the edges, the metal belt can be relieved of a major temperature difference otherwise existing along the web edge, as well as of stress conditions resulting therefrom and possibly causing fatigue fractures.

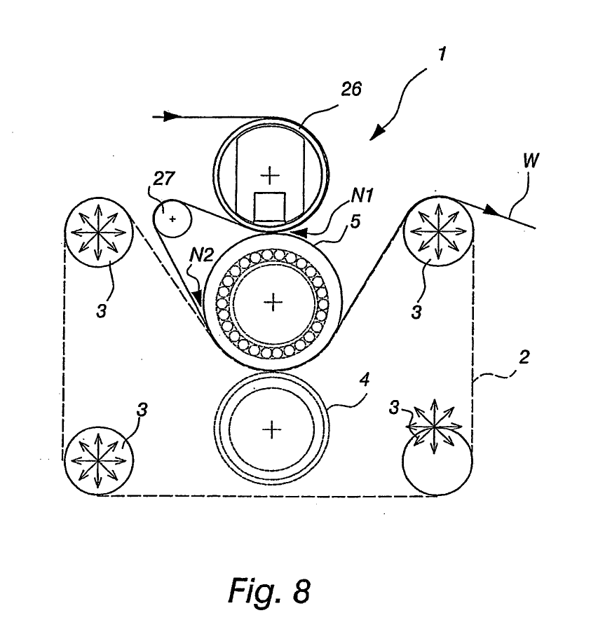

FIG. 8 shows one solution of the invention, wherein a fibrous web W is first guided through a profiling nip N1, established by a deflection-compensated nip roll 26 disposed outside the belt loop of a metal belt calender 1 with a roll 5, and then passed over a guiding element 27 to a nip N2 between the roll 5 and a belt 2.

The arrangement of FIG. 8 is particularly suitable for boards as one and the same machine is used for producing coated and uncoated grades. Another idea in this embodiment of a processing device is that, in addition to a tension of the belt 2, it is also possible to adjust an overlap angle of the belt in a nip, i.e. the length of a nip. This enables running higher-thickness grades with a shorter nip as the speed is lower, and lower-thickness grades with a longer nip. Thus, diverse-thickness boards have only the topliner heated and plasticized, and the thermal energy consumption of a thermal roll will be optimized at the same time.

The roll 5 comprises preferably a thermal roll. Instead of a single roll 5 depicted in FIG. 8, the number of thermal rolls can be possibly even up to three. On the other hand, the deflection-compensated nip toll 26 may comprise a chilled-surface or preferably for example a composite-shelled, rubber-covered, deflection-compensated roll. This results in a good profiling capability, and the nip is capable of calendering, without instability of gloss, those grades which are not coated after calendering.

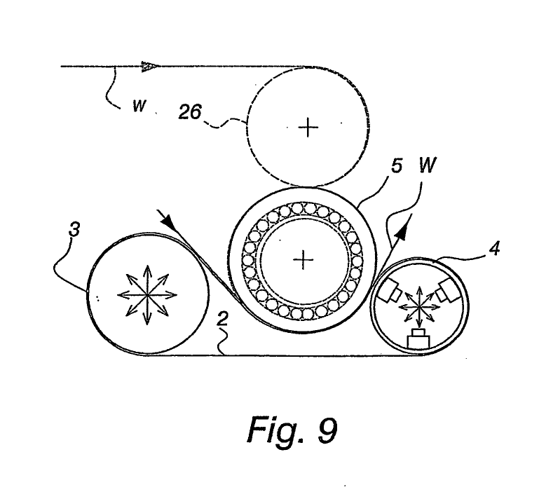

According to one preferred embodiment of the invention, it is also possible to employ a press element disposed inside a belt loop, particularly a roll 4. Such a preferred solution is depicted in FIG. 9, wherein the roll 4 is designed as a shoe roll and establishes at the same time with a roll 5 an extra nip, which can be located within or upstream or downstream of the confines of a nip N2 established by the roll 5 and a belt 2. The roll 4 can be preferably made movable the same way as a guiding element 3, such that, if desirable, it can be used for tightening the belt 2.

It should be noted that a web W can be adapted to travel in the device in either direction. Profiling can also be provided both upstream and downstream of the nip. It is conceivable, for example, that FIG. 9 be provided with a dash-line designated, deflection-compensated nip roll 26 to establish a profiling nip with the roll 5 as in the exemplary embodiment for a device shown in FIG. 8.

It should also be pointed out that the rolls 4, 5 and 26, as well as the guiding elements 3, can be mounted on a common frame or separate frames. In particular, it should be noted that various embodiments of the invention, such as, for example, in FIGS. 8 and 9, can be set in various angular positions. For example, it is possible to achieve a zero nip load by means of an appropriate relative disposition/appropriate angular positions of components.

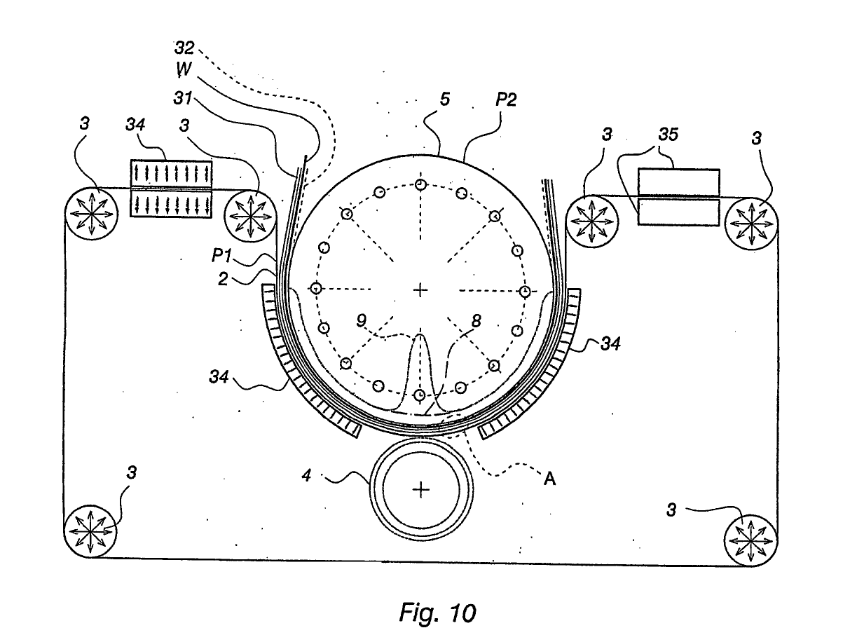

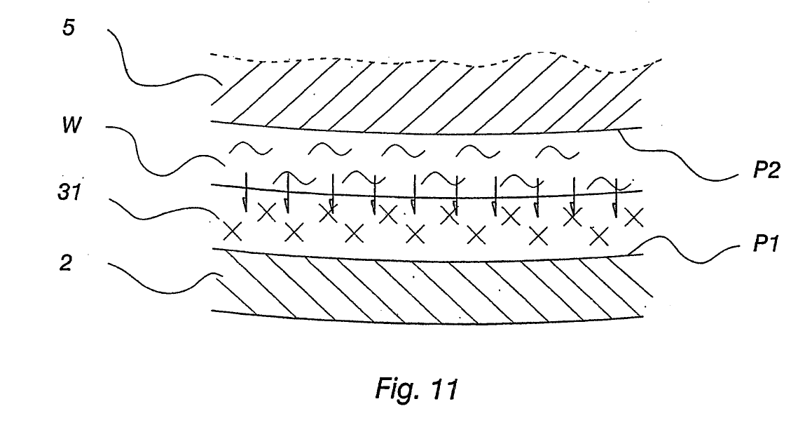

FIG. 10 illustrates one device of the invention for drying a fibrous web by means of condensation drying, said device comprising a metal-constructed belt 2, which forms a surface P1 and extends around a guiding element 3. The belt 2 is further adapted to extends around a counter-element 5, which is disposed outside the same and which forms a surface P2. The belt 2 and the roll 5 establish therebetween a drying zone for passing a to-be-dried fibrous web W therethrough. In addition, between the belt 2 and the counter-element 5 is adapted to advance at least one porous, air permeable wire 31, such that, in this embodiment of the invention, the fibrous web W is in contact with the surface P2 heated in the drying zone, and the wire 31 is in contact with the cooled surface P1. The arrangement is shown in more detail in FIG. 11.

The fibrous web W to be dried travels through the drying zone, being subjected to a desired pressure impulse and thermal effect as a function of time. In response to the heated surface P2, the temperature of water contained in the fibrous web W rises and the water vaporizes. The vaporized water migrates through the porous wire 31 onto the cooled surface P1 formed by the belt 2 for further condensation thereon.

In a highly preferred embodiment of the invention shown in FIG. 10, the surface P2 is heated and P1 is cooled. The counter-element 5 forming the surface P2 may most conveniently comprise a thermal roll, which is the case in the embodiment of FIG. 10. Heating and temperature control of the roll 5 are readily feasible for the purpose of the invention. All prior known heating solutions for a conventional thermal roll can be used. It is also conceivable to use a device of the invention in the dryer section of a paper or board machine, whereby the roll 5 functioning as a counter-element comprises a conventional drying cylinder. In this case, heating and temperature control of the roll 5 are performed in a prior known manner by means of steam and by regulating its pressure.

In the configuration of FIG. 10, the surface P2 is formed by a dash-line designated metal belt 32, adapted to extend around the roll 5. Thus, heating of the surface P2 can, on the one hand, be effected by heating the belt 32 alone or, on the other hand, by the application of heating both to the belt 32 and to the roll 5. The application of heating to the roll 5 as well provides a better-than-before control over temperature of the belt 32 and the consistency thereof throughout the drying zone as it is in contact with the roll 5 for further enhancing the drying process of a fibrous web. A variety of options are available for heating the belt 32 and those will be described in more detail later in conjunction of the exemplary embodiment of FIG. 3, which deals with alternative heating means for the belt 2.

Respectively, the belt 2, functioning as the cooled surface P1, can have its cooling effected in a plurality ways, for example by heat transfer to a cooling liquid, to an evaporating surface, to a cooling roll or belt. In FIG. 10, the cooling means applied for cooling the belt 2 are indicated with numeral 34. It is conceivable that the belt 2 be cooled, for example, with a cold water jet, a cold air injection or by some other prior known method. It is likewise conceivable that the belt 2 be provided in a prior known manner with dehumidifiers 35.

Thus, according to the invention, in order to adjust the belt 2 to a desired tension, at least some of the guiding elements 3 are adapted to be movable. In addition to the tightness of the belt 2, a contact pressure applied to a fibrous web in the drying zone can be further increased and, at the same time, the composition of a fibrous web itself can be influenced by fitting inside the belt 2 additionally at least one press element 4 for compressing the belt 2 against the counter-element 5. In one preferred embodiment of the invention, the press element comprises at least one roll 4. The roll 4 may also be deflection-compensated and it can be selected from a group, including an elastic surface roll, such as a polymer-covered roll, a rubber-covered roll or an elastomer surface roll, a shoe roll, a thermal roll, a metal roll, a filled roll, and a composite roll. The roll 4 can also be shiftable for varying the processing zone length and/or belt tension.

A dash-and-dot line 9 in FIG. 10 represents the pattern of a pressure impulse in the case when inside the belt 2 is fitted a roll 4 functioning as a press element, in this case a nip roll which compresses the belt 2 against the roll 5 to establish a higher contact pressure within the drying zone. A dash line 8, in turn, represents the pattern of a pressure impulse in the case when the contact pressure existing in the drying zone is established solely by means of a tension of the belt 2, the nip roll 4 being out of a compressive contact with the belt 2 or with no nip roll 4 at all fitted inside the belt 2.

The other main operations in the process, with regard to venting a web and wires/felts, removal of condensate from a wire, as well as supporting the passage of a web and wire, can be implemented basically as described in the preambles of the cited publications FI 97485 B1 and FI 99272 B1, and, thus, shall not be explained further at this time.

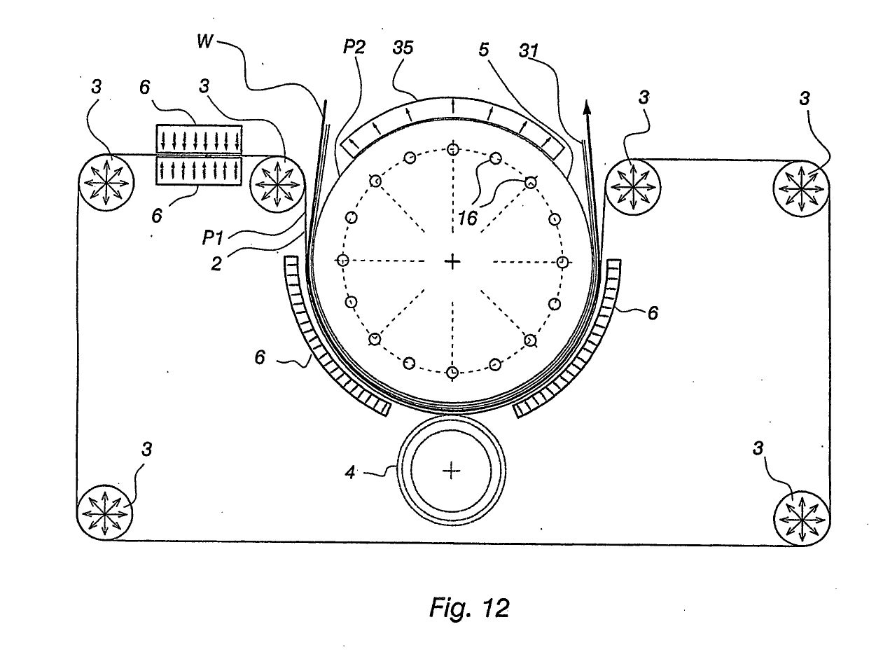

In another preferred embodiment of the invention shown in FIG. 12, the belt 2, which forms a surface P1, is heated and the counter-element 5, which forms a surface P2, is cooled, respectively.

In this embodiment, a fibrous web W is in contact with a belt 2, which forms a surface P1, while a porous wire 31 is in contact with a roll 5, which functions as a cold surface P2. The surface of the roll 5 itself can also be made porous, whereby it actually replaces the porous wire 31 and a separate wire is not necessarily needed. The roll 5 can also be provided with a hard or soft surface. Furthermore, the belt 2 and/or the roll 5 can be smooth or embossed in its surface and the contact surface established by the belt and/or the roll with a web W can travel at a speed different from that of the web W.

Heating of the belt 2 is feasible, for example, by means of induction or some other prior known means. In a particularly preferred case, the belt 2 can have its heating provided by means of guiding elements 3. The guiding elements 3 can be heated by any prior known heating method, preferably from inside, with water, steam, or, as especially preferred, with oil or internal combustion. The belt 2 can have its heating provided also by means of separate heating units, represented by reference numeral 6, such as, for example, an induction heater, an infrared radiator, or a gas burner.

The surface P2 or counter-element 5 can have its cooling handled, for example internally, by the application of the heat transfer principles of a thermal roll, by circulating a cooling heat carrier in an intra-roll manifold 16. It is also conceivable that vaporization of a suitable refrigerant be effected inside the roll 5, i.e. the roll 5 functions as a component in a heat pump process, whereby the recovered heat could be used elsewhere. In association with the roll 5 are preferably provided dehumidifiers 35.

In this embodiment, it is also possible to arrange inside the belt 2 at least one roll 4. which functions as a press element and provides a nip with the counter-element 5. A fibrous web can be thereby subjected to an extra contact pressure within the nip zone, and hence to have an enhanced effect also on the composition and properties of the fibrous web. The elevated temperature, together with a long application time and a wide-range pressure control capability, offers a possibility of applying a beneficial effect on a fibrous web both at high and low speeds, e.g. at speeds of 100 m/min to 4000 m/min.

In this embodiment of the invention, The other main operations in the process, with regard to venting a web and wires/felts, removal of condensate from a wire, as well as supporting the passage of a web and wire, can be implemented also basically as described in the cited publications FI 97485 B1 and FI 99272 B1, and shall not be explained further at this time.

In order to establish a contact area, formed by the counter-element 5 with the belt 2, and a fibrous web drying zone, it is possible to use counter-elements in a variety of configurations. As in the exemplary embodiments shown in FIGS. 1 and 3, the counter-elements and press elements can be rotating or non-rotating rolls. These can additionally be provided with a crowning for controlling a lateral or crosswise tension of the web. It is also conceivable that the counter-element comprises something other than a roll, such as, for example, various support bars. Furthermore, by using diverse press elements, it is possible to have a desired effect on the pattern and magnitude of a pressure impulse applied to a fibrous web in the drying zone.

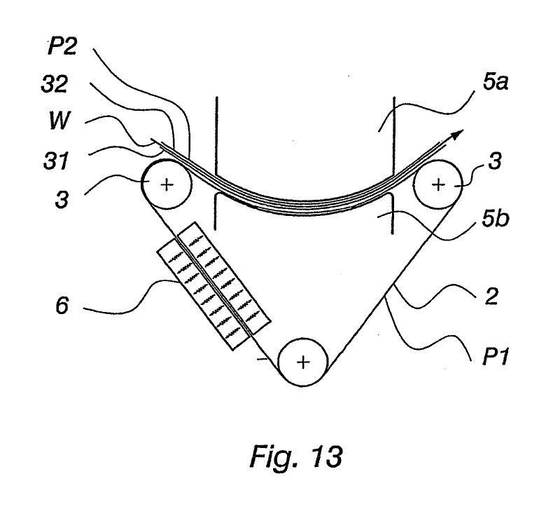

In an embodiment of the invention, implemented by means of fixed bar elements and shown in FIG. 13, heating can be applied to the surface P2 and cooling to the surface P1, or vice versa. In the embodiment of FIG. 13, a belt 32, functioning as the surface P2, is heated and a belt 2, functioning as the surface P1, is cooled. In order to facilitate temperature control over the belt 32, heating can also be additionally applied to a bar element 5a, or optionally omitted. Likewise, the surface P1 can have its cooling applied solely to the belt 2, or additionally also to a bar element 5b. The heating and cooling methods may comprise all of the above solutions.

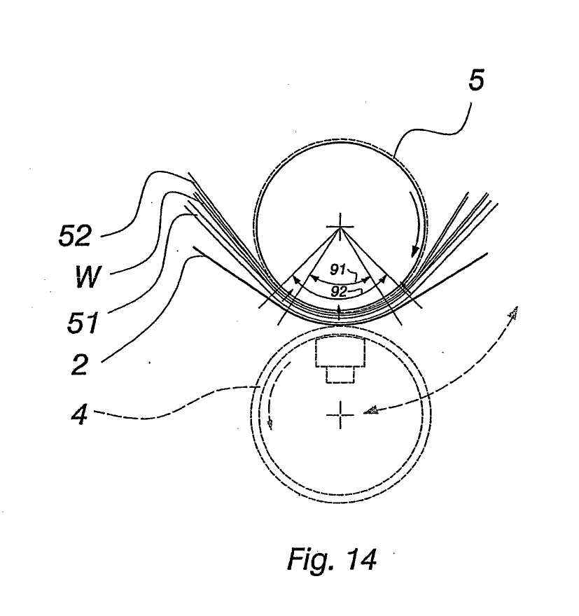

FIG. 14 illustrates a portion of a device of the invention, which comprises a metal belt 2 as described above and intended for pressing, and especially for drying a fibrous web W, and which is provided between the belt 2 and the web W with a porous felt 51, as well as between the web and a counter-element 5 with a felt 52, respectively.

In the process of being conveyed into a pressing zone, the fibrous web W is subjected to a compression force and, as the web compresses, the water contained therein migrates, being driven by pressure loads, from the web into the surrounding felts/wires and remains therein after the pressing zone. The leading concept in a solution of the invention is to exploit the above-described processing device for extending the pressing zone and, consequently, to lengthen the application time of a press. Thus, the belt loop is used for setting a fibrous web in a substantially longer-lasting compressive contact with a counter-element. At the same time, the length and pressure of a contact area can be controlled more easily than before. Likewise, the opening stage can be controlled better and, thus, delamination is avoided.

A function of the felts is to receive an ingredient migrating thereto from a fibrous web, in this case, water. Another function of the felts is to operate as a flexible element within the pressing zone and to support a web elsewhere outside the pressing zone. The pressing zone can either be provided with a single felt lining, the felt being only used on one side of a web W, or with a double felt lining, the felt being used on both sides of the web W. It is also conceivable that more than one felt be provided on one side of the web. It is further conceivable that, instead of a felt, the web be provided on one side or on both sides with an appropriate type of wire.

A belt loop is supported and guided by means of separate guiding rolls 3 (e.g. as shown in FIG. 1). The location and position of one or more guiding rolls 3 can be adjusted. What is essential in this respect is that the passage of a belt loop is modifiable by means of the guiding rolls 3 in such a way that the length of a pressing zone (“overlap angle”) can be readily controlled. Likewise, the opening stage can be controlled in such a way that delamination of a web W cannot happen. The belt 2 in a belt loop may comprise a metal belt or a composite metal belt.

In this case, as well, the belt loop is used for setting a fibrous web W in a substantially longer-lasting compressive contact with a counter-element 5. The length and pressure of a contact area are also more easily controllable than before. Likewise, the opening stage can be controlled better to thus avoid delamination. Pressure control in a pressing zone can be further improved by regulating individually the pressure effect of a belt 2 as well as possibly added belt loops lying inside or on top of each other, i.e., in practice, by adjusting a tension thereof independently of each other. In addition, the opening points of various belt loops can be adapted to occur successively at appropriate intervals, as visualized e.g. in FIG. 14. The belt 2 establishes a pressure zone 91 and the felt or the wire 51, the latter being especially preferred in this case, establishes a pressure zone 92.

Inside a belt loop there may be one or more support elements 4 for each counter-element 5, the purpose of which is to increase a compression load resulting from the tension of a belt 2. Thus, the pressure in a pressing zone between the counter-element 5 and the belt loop results from a tension of the belts 2 and the felts/wires 51, 52, and from possible extra loads created by the support elements 4.

It is further possible to use the counter-element 5 for taking up the water migrating from the web W or the felts 51, 52. Especially in this case, the counter-roll 5 may comprise a suction roll or some other porous or perforated or grooved-surface roll.

Thus, the web W can set in a direct contact with the counter-element 5, and the felts or wires, represented by numeral 52, can be omitted completely. In special applications, it is further possible to use embossed and/or engraved rolls or belts, should the fibrous W be provided with some sort of pattern.

One of the main functions of the counter-element 5 is to operate both as a bearing surface and possibly also as a dewatering means. It is further conceivable to use the counter-element 5 as a heat source, such as in a so-called hot-pressing and impulse-drying process, which is known from numerous publications. The counter-element may comprise a thermal or press roll (hard, ceramic-covered, or porous surface), a belt roll (shoe roll), a deflection-compensated roll, an elastic surface roll (rubber, polymer, etc.), a composite roll, or the like. The counter-roll may also comprise another belt loop, such as a metal or polymer belt, a wire, or the like, which is respectively supported from inside the belt loop by means of a support element.

The counter-element 5 can also be heated. Thus, heating reduces the viscosity of water, possibly vaporizes water, and hence generates a steam pressure effect propelling water towards the felt. In addition, the heating of a web supplies the same with thermal energy which, as the pressure drops in a nip opening stage, results in powerful vaporization (so-called flashing).

The belt 2, the fibrous web W, the wires and/or felts 51, 52, as well as the support element 4 can also be heated or cooled. Heating can be carried out by conventional means. The belt 2, for example, can be heated by induction.

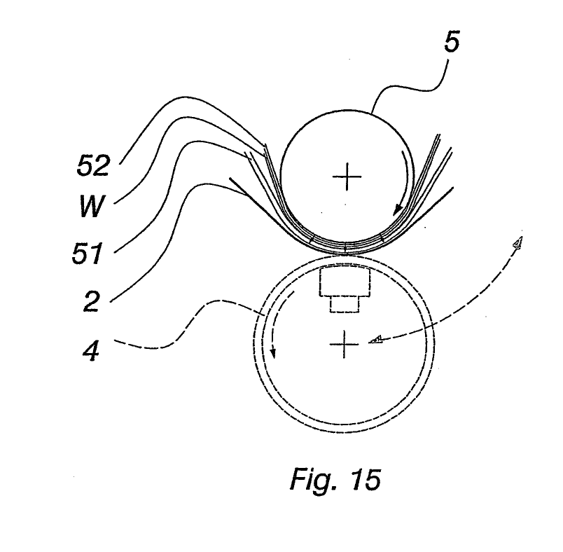

In a particularly preferred case, if the felt is located “on the outside” with respect to the curvature of a web, the result is a water-propelling centripetal force assisting in the migration of water. At high speeds and with small roll radii, the centripetal force can be so significant that the actual pressing action can be reduced or even omitted. At the very least, the centripetal force works against so-called rewetting, i.e. impedes a back flow of water from felt to web. FIG. 15 depicts an embodiment of the invention, wherein a water-accepting felt 51 is provided between a web W and a belt 2 and wherein the migration of water from the web W to the felt 51 is enhanced by means of a centripetal force. The effect has been further intensified by making the diameter of a roll 5 smaller than that of the roll 5 in the embodiment of FIG. 14. In the process of reducing the size of a roll 5, it is necessary to consider the bending strength requirements that must be satisfied by the belt 2 and the felts/wires.

In association with wire and felt loops are provided necessary washing and drying functions, as known from current solutions.

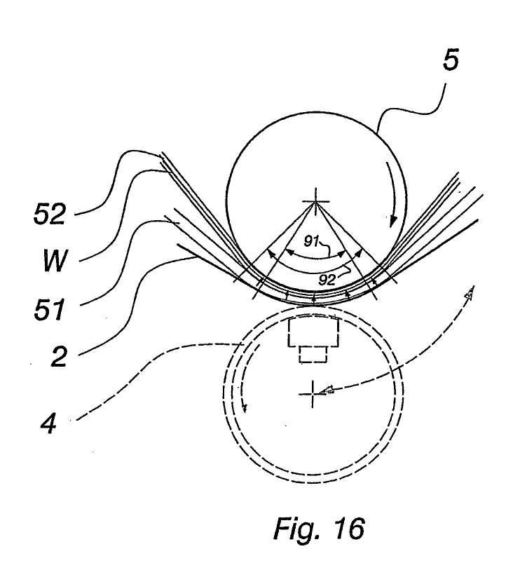

FIG. 16 illustrates a device according to this further aspect of the invention for drying a fibrous web W, comprising, in addition to the web, at least one belt or wire 2 capable of attaining a tension and forming a dense bearing surface, and at least one particularly porous and compressible felt/wire 51. On the opposite side of the web W lies a water-accepting wire/felt 52, which is porous but substantially less compressible than the first felt 51.

In the process of leading the fibrous web W and the felts 51 and 52 into a pressing zone between a counter-element 5, in this case a roll, and a belt 2, the felt 51 first compresses, whereby a gas contained in its pores compresses and a pressure in the felt increases. As the pressure seeks to equalize or level out, the gas flows towards the web W and propels water present in the web pores in front of it, forcing it to migrate into the felt 52. In this respect, the phenomenon is similar to what happens with an impulse press, but what is essential is that the pressure is provided by mechanical compression instead of vaporization.

What is essential is to dimension the pore volume and compressibility of felts relative to each other, appropriately with respect to both the web W and the applied forces/pressures for providing a desired pressure difference and dewatering action.

Pressure control in a pressing zone can be further improved by regulating individually the pressure effect of a belt 2 as well as possibly added belt loops lying inside or on top of each other, i.e., in practice, by adjusting a tension thereof independently of each other. Likewise, the occurrence of delamination can be impeded by adapting the opening points of belt loops to take place successively, preferably at appropriate intervals. This is visualized in FIG. 16 by pressure zones 91 and 92 established by belt loops 2 and 51.

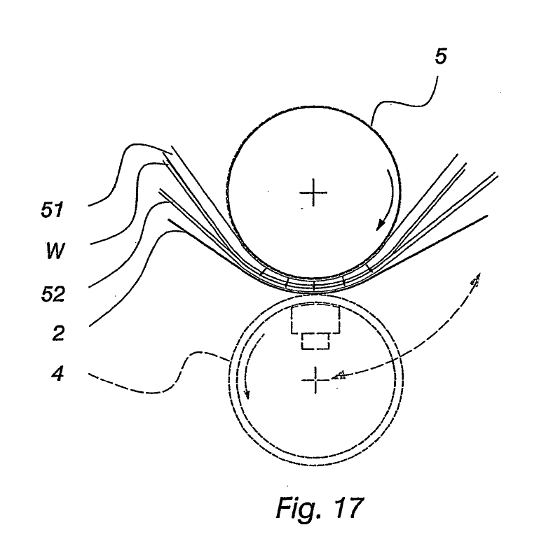

In the embodiments of both FIG. 16 and 17, the compression can be enhanced by means of an extra loading roll 4, which can be adapted to be movable for varying the length of a processing zone and/or the tension of a belt 2. The roll 4 may also comprise a profiled roll. Moreover, in association with the belt and felt/wire loops are provided necessary washing/drying actuators. Furthermore, in the arrangement of FIG. 16, the roll 5 may preferably comprise a suction roll, whereby the suction effect can be used for further enhancing the dewatering action as the pressure difference increases. Thus, the numbered felt in FIG. 16 can be omitted completely.

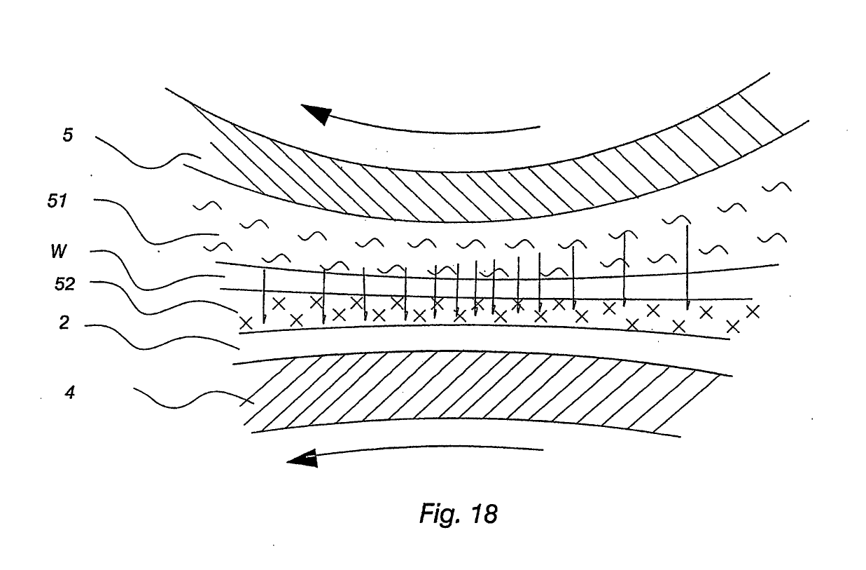

In this embodiment of the invention, as well, the roll/belt loop system can be preferably designed in such a way that there is a centripetal force assisting in the migration of water in a radial direction of the roll 5, as illustrated in FIG. 17. Thus, the felt 51, which is more compressible and from which air migrates towards a web W, is arranged between the roll 5 and the web W on “the inside curve”. As air migrates from the felt 51 towards the web W and forces the water contained in the web to migrate into a felt 52, the centripetal force further enhances this effect. FIG. 18 is a schematic close-up, illustrating a detail in the exemplary embodiment of FIG. 17 from the region of a nip established by the extra loading roll 4. The figure visualizes the enhancing effect of a gas contained in the pore volumes of felts for drying the fibrous web W within a pressing zone.

The following description deals with a fibrous web sizing process, with reference to the mechanical configuration shown in FIGS. 1-7.

When the question is about a stock or internal sizing process, the sizing agents are preferably admixed within stock by some conventional means at the wet end upstream of the headbox of a paper machine, and, thus, it is not described further in this context. On the other hand, when the question is about surface or top sizing, the sizing agents can be applied to the surface of a fibrous web W during the course of a manufacturing process in a prior known manner, not described further in this context, either on-line or off-line, for example by using film transfer technology, by spraying or brushing. Application is preferably effected just before passing the fibrous web W into a processing zone in a metal belt calender.

Sizing can be preferably effected by using sizing agents, such as resins obtained from softwood pitch, as well as synthetic AKD (alkylketene dimer) and ASA (alkenyl succinic anhydride) sizes. Wet strength sizes are used for enhancing wet tensile strength, and dry strength sizes are used for reinforcing the texture of dry paper. Dry strength sizes comprise, for example, starch and the above-mentioned synthetic sizes.

A fibrous web W to be treated is passed through a processing zone, being subjected to a desired pressure impulse and thermal effect as a function of time. In the process of heating and compressing the fibrous web W in a contact zone, the sizing agents are finally bonded to fiber surfaces. In addition, the web develops new fiber bonds. Lignin, in particular, develops new bonds in addition to forming simultaneously a layer protecting fibers from water. What is essential in a process of the invention is that the application time for treating a fibrous web in a processing zone be sufficient, about 10-300 ms. The bonding of sizing agents to fiber surfaces is enhanced by raising the temperature of the fibrous web W to a sufficiently high level. Reference numeral 6 (FIG. 1) represents heating means for heating the metal belt 2, such as an induction heater, an infrared radiator, or a gas burner. Heating can be based also on resistive heating. A solution of the invention can be implemented by using elevated temperatures, for example from about 200° C. to as high as about 400° C., depending on the thickness, moisture, and other properties of paper or board to be processed, sizing agents applied, as well as processing time.

The optional implementations for a processing device shown in FIGS. 1-7 can be preferably used for treating a fibrous web W with sizing agents. The pressurized thermal treatment of a fibrous web for enhancing a sizing process can be conceivably effected, according to the invention, also in the dryer section of a paper or board machine, in which case the belt comprises a metal belt, and the counter-element establishing a contact area therewith comprises a drying cylinder. It is further conceivable that the treatment of a fibrous web for enhancing the effect of sizing agents be performed for example with a shoe press or a CondeBelt-type of arrangement, wherein two metal belts are adapted to travel in contact with each other over a certain distance.

Furthermore, particularly in the case of surface sizing, the eventual moisture control of a fibrous web can be performed by conventional means, for example by steaming the web surface/surfaces prior to leading the web into a processing zone. Moistening and/or temperature control can be used for an impact on the sizing process and, thus, the method provides a possibility for wide-range fluctuation of web moisture.

According to the invention, a feasibility is offered for providing a concurrent sizing and calendering action. A metal belt calender of the invention can be operated at considerably high speeds and also at an elevated temperature. The elevated temperature, together with a long application time and a wide pressure control range, can be used for providing at the same time a good calendering result at both high and low speeds, e.g. at speeds of 100 m/min to 4000 m/min. In addition, the inventive metal belt calender provides a supported web passage through a processing zone and allows for a controlled variation of web width within the range defined by belt width. Web feeding is feasible over a full web width and at a high web speed.

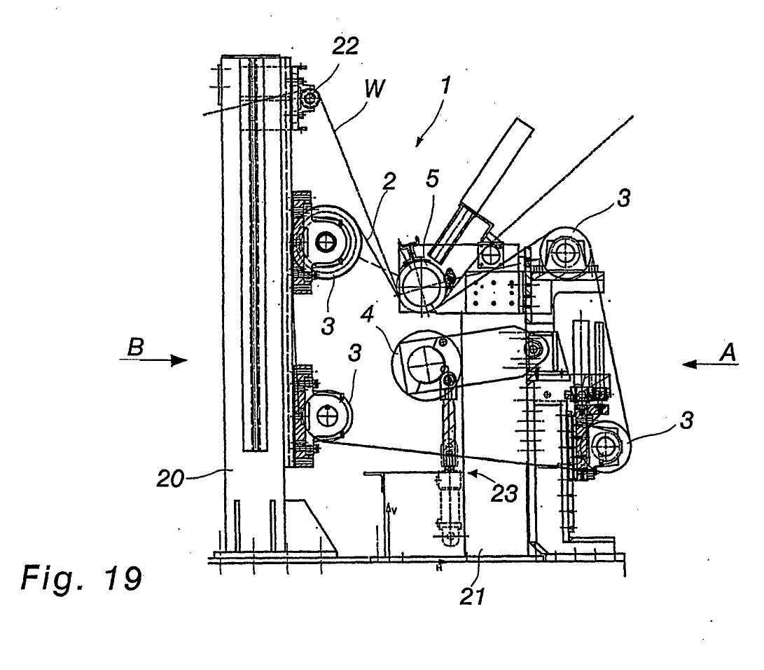

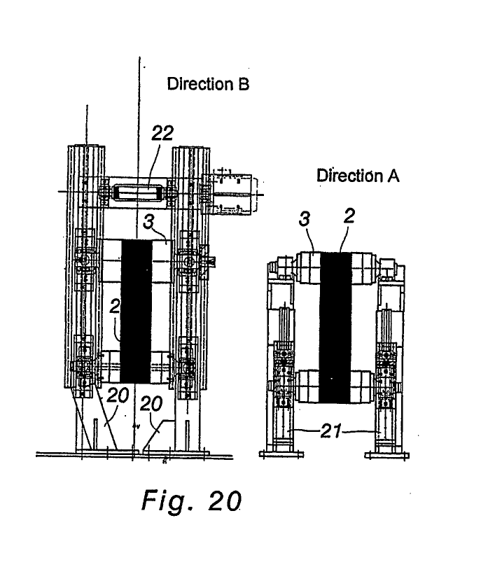

FIGS. 19 and 20 illustrate schematically one pilot machine of the invention in side and end views, respectively, the corresponding components being indicated by the same reference numerals as in the preceding figures. Reference numeral 20 represents a first vertical frame of the pilot device, on which are mounted, with a per se known bearing assembly, first guiding rolls 3 for a belt 2. On the vertical frame 20 is further mounted, with a per se known bearing assembly, a guide roll 22 for a web W. Reference numeral 21 represents a second vertical frame for a pilot device 1, on which are mounted second guiding rolls 3 for the belt 2, as well as a counter-roll 5 and a press roll 4. A processing zone is established between the belt 2 and the counter-roll 5 and the web 5 is passed through said processing zone. The press roll 4 remains inside the belt loop and is contactable by means of loading elements 23 with the inner surface of the belt 2 for establishing, together with the counter-roll 5, a higher-pressure nip area within the processing zone.

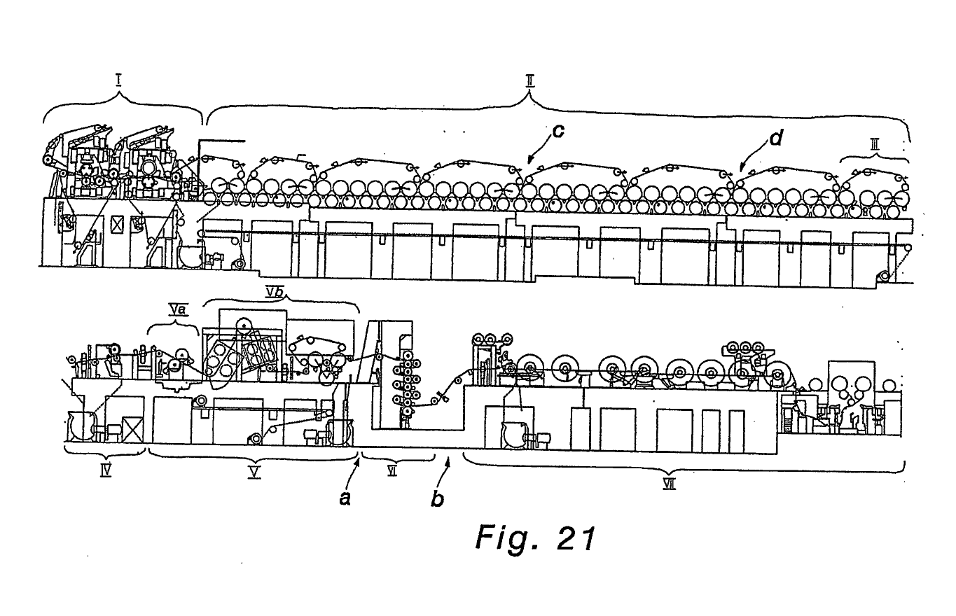

FIG. 21 depicts one embodiment for an LWC paper production line, showing the line sections downstream of a press section I. The press section is followed by a dryer section II, the tail end portion of which is indicated by reference symbol III. The dryer section is followed by a precalendering process IV and then by a coating process V, which is divided into a coating station Va and a drying portion Vb. The coating station is followed by a final calendering process VI and ultimately by finishing processes VII, including e.g. winding operations. It is conceivable for a processing device of the invention to be located in an on-line production line for LWC paper, e.g. as indicated by reference symbols a, b, c and/or d. In addition to or instead of these locations, it is conceivable that a processing device of the invention be used for replacing, for example, the tail end portion III of a dryer section and/or the precalender IV and/or the final calender VI, the wet press I, the sizing device Va, or e.g. the coating device Vb.

Generally speaking, it can be concluded that the inventive processing device provides a very high efficiency for calendering and/or other treatment in a single processing zone. The inventive device allows for very extensive ranges for pressure, temperature and dwell times, which are combinable in a variety of ways depending on a particular application. For example, a pressure domain can be within the range of about 0.01 MPa to about 70 MPa or even up to the reading of about 200 MPa, temperature can be within the range of about −70° C. to about +400° C., and a dwell time in the processing zone e.g. within the range of about 0.01 ms to about 2 s, or even in the order of about 10 s. Moreover, various grades can be manufactured by using various machine speeds. The inventive device may comprise an on-line or off-line device.

1. A processing device for processing at least one of a coated fibrous web and an uncoated fibrous web, comprising at least one of a metal belt and a metal-based belt configured to extend around at least one guiding element, at least one counter-element being disposed outside said belt to provide a contact area with the belt, such that the belt and the counter-element establish therebetween a web processing zone for passing the web to be processed therethrough, wherein a length of the processing zone is defined by at least one of the disposition of the guiding element and the configuration of said at least one counter-element, wherein a processing time of the web in the processing zone is within the range of between about 10 ms and about 1000 ms, and wherein a contact pressure applied to the web in the processing zone is within the range of between about 0.01 MPa and about 200 MPa.

2. A device as set forth in claim 1, wherein the processing device comprises at least one of a calender, a coater, a film transfer device, a printing device, a drier, and a press.

3. A device as set forth in claim 1, wherein the counter-element comprises a roll selected from the group consisting of a deflection-compensated roll, an elastic surface roll, a polymer-covered roll, a rubber-covered roll, an elastomer surface roll, a shoe roll, a thermal roll, a metal roll, a filled roll, and a composite roll.

4. A device as set forth in claim 3, wherein the roll comprises a thermal roll, and wherein an operating temperature of at least one of the thermal roll and the belt is within the range of between about 50° C. and about 400° C.

5. A device as set forth in claim 3, wherein the roll comprises a thermal roll, and wherein the operating temperature of at least one of the thermal roll and the belt is within the range of between about −70° C. and about +50° C.

6. A device as set forth in claim 4, wherein said operating temperature is higher than about 200° C.

7. A device as set forth in claim 4, wherein the operating temperature is within the range of between about 250° C. and about 300° C.

8. A device as set forth in claim 1, wherein the belt has a thickness of between about 0.1 mm and about 3 mm and a tensile stress within the range of between about 10 MPa and about 500 MPa.

9. A device as set forth in claim 8, wherein a thickness of the belt is within the range of between about 0.3 and about 1.5 mm.

10. A device as set forth in claim 1, wherein at least one press element is provided inside the belt for pressing the belt against the counter-element.

11. A device as set forth in claim 10, wherein the press element comprises at least one roll selected from the group consisting of a deflection-compensated roll, an elastic surface roll, a polymer-covered roll, a rubber-covered roll, an elastomer surface roll, a shoe roll, a thermal roll, a metal roll, a filled roll, and a composite roll.

12. A device as set forth in claim 11, wherein the roll is shiftable for at least one of varying the length of the processing zone, varying a tension of the belt, and adjusting a magnitude and location of a pressure effect in the processing zone.

13. A device as set forth in claim 11, wherein the counter-element comprises a second belt adapted to be driven around guiding elements, the processing zone being established between the two belts.

14. A device as set forth in claim 1, wherein the press element comprises a second belt loop inside the belt.

15. A device as set forth in claim 1, wherein the belt comprises at least one of a steel-reinforced rubber belt and a covered belt.

16. A device as set forth in claim 1, wherein the belt has an embossed surface for providing a desired pattern on the web.

17. Use of a device as set forth in claim 1 as at least one of a precalender upstream of a coating process, a final calender downstream of a paper machine, a final calender downstream of a coating process, a breaker stack, a drying device, a coating device, a sizing device, a printing device, and a cooling device.

18. A method for processing at least one of a coated fibrous web and an uncoated fibrous web with a processing device, the processing device comprising at least one of a metal belt and a metal-based belt configured to extend around a guiding element, at least one counter-element being disposed outside said belt to provide a contact area with the belt, such that the belt and the counter-element establish therebetween a web processing zone for passing the web to be processed therethrough, wherein the method comprises defining a length of the processing zone by at least one of the disposition of the guiding element and by the configuration of the counter-element, processing the web in the processing zone for between about 10 ms and about 1000 ms, and adjusting a contact pressure applied to the web in the processing zone to within the range of between about 0.01 MPa and about 200 MPa.

19. A method as set forth in claim 18, wherein the method uses at least one press element provided inside the belt for pressing the belt against the counter-element for enhancing a pressure pulse applied to the web passing through the processing zone.

20. A method as set forth in claim 18, wherein the counter-element used in the method comprises a thermal roll having a temperature that is raised for processing the web to an elevated temperature within the range of between about 70° C. and about 400° C.

21. A method as set forth in claim 18, wherein the belt has a temperature that is raised for processing the web to an elevated temperature within the range of between about 150° C. and about 400° C.

22. A method as set forth in claim 20, wherein the temperature of at least one of the thermal roll and the belt is raised for processing the web to an elevated temperature higher than about 250° C.

23. A method as set forth in claim 18, wherein at least one of the belt and the counter-element is cooled to a temperature of between about −70° C. and about +50° C.

24. A method as set forth in claim 18, wherein a pressure pulse applied to the web passing through the processing zone has a pattern adjusted by at least one of a tension of the belt, the configuration of the press elements, a compression force applied to the web by the press elements, and moving the press element.

25. A device as set forth in claim 1, wherein the device comprises a cleaning device provided downstream of the processing zone for cleaning the belt over the paper web-facing surface thereof.

26. A device as set forth in claim 1, wherein the belt is heated and the device is provided with a cooling device for cooling the heated belt, along edges thereof, with at least one of a water injection arrangement and an air injection arrangement for eliminating high temperature differences in the belt along the edge thereof.

27. A processing device for processing at least one of a coated fibrous web and an uncoated fibrous web, comprising at least one of a metal belt and a metal-based belt configured to extend around a guiding element, at least one roll being disposed outside said belt to provide a contact area with the belt, such that the belt and the roll establish therebetween a web processing zone for passing the web to be processed therethrough, wherein outside the belt is provided a deflection-compensated nip roll to establish a profiling nip with the roll, with the web being configured to travel through said profiling nip, wherein a length of the processing zone is defined by at least one of the disposition of the guiding element and the dimensioning of the roll, wherein a processing time of the web in the processing zone is within the range of between about 10 ms and about 1000 ms, and wherein a contact pressure applied to the web in the processing zone is within the range of between about 0.01 MPa and about 200 MPa.

28. A device as set forth in claim 27, wherein the processing device comprises at least one a calender, a coating device, a sizing device, a printing device, a drier, and a press.

29. A device as set forth in claim 27, wherein the profiling nip is established both upstream and downstream of the processing zone, the web being configured to travel through the profiling nip both upstream and downstream of the processing zone.

30. A device as set forth in claim 27, wherein the roll comprises a deflection-compensated roll.

31. A device as set forth in claim 27, wherein the roll is selected from the group consisting of a deflection-compensated roll, an elastic surface roll, a polymer-covered roll, a rubber-covered roll, an elastomer surface roll, a shoe roll, a thermal roll, a metal roll, a filled roll, and a composite roll.

32. A device as set forth in claim 31, wherein the roll comprises a thermal roll and an operating temperature of at least one of the thermal roll and the belt is within the range of between about 50° C and about 400° C.

33. A device as set forth in claim 32, wherein said operating temperature is higher than about 200° C.

34. A device as set forth in claim 32, wherein the operating temperature is within the range of between about 250° C. and about 300° C.

35. A device as set forth in claim 27, wherein the belt has a thickness of between about 0.1 mm and about 3 mm and a tensile strength within the range of between about 10 MPa and about 500 MPa.

36. A device as set forth in claim 35, wherein the belt thickness is within the range of between about 0.3 mm and about 1.5 mm.

37. A device as set forth in claim 27, wherein inside the belt is provided at least one press element for pressing the belt against the roll.

38. A device as set forth in claim 37, wherein the press element comprises at least one roll selected from the group consisting of a deflection-compensated roll, an elastic surface roll, a polymer-covered roll, a rubber-covered roll, an elastomer surface roll, a shoe roll, a thermal roll, a metal roll, a filled roll, and a composite roll.

39. A device as set forth in claim 27, wherein the roll comprises a second belt configured to be driven around guiding elements, such that the processing zone is established between the two belts.

40. A device as set forth in claim 27, wherein the belt comprises at least one of a steel-reinforced rubber belt and a coated belt.

41. A device as set forth in claim 27, wherein the belt has an embossed surface for providing a pattern on the web.

42. A device for drying a fibrous web, said device comprising at least one of a dense, air impermeable metal belt and a dense, air impermeable metal-based belt, defining a surface and configured to extend around at least one guiding element, at least one counter-element defining a surface and being disposed outside said belt to provide a contact area with the belt, the surfaces establishing therebetween a fibrous web drying zone for passing the to-be-dried fibrous web through said drying zone, wherein one of the surfaces is heated and the other is cooled, at least one porous wire being further configured to travel between the surfaces such that the fibrous web is in contact with the heated surface and the wire is in contact with the cooled surface, any steam escaping from the fibrous web migrating through the wire to the cooled surface and condensing thereon, wherein the belt extends about the counter-element in compliance with the surface thereof, wherein a length of the drying zone is configured to be adjustable by at least one of the disposition of the guiding element and the configuration of the counter-elements, wherein a drying time of the web in the drying zone is within the range of between about 10 ms and about 1000 ms, and wherein a contact pressure applied to the fibrous web is within the range of between about 0.01 MPa and about 70 MPa, such that the contact pressure applied to the fibrous web in the drying zone is at least equal to a steam pressure generated by vaporization of water contained in the fibrous web.

43. A drying device as set forth in claim 42, wherein the counter-element is heated and the belt is cooled.

44. A drying device as set forth in claim 42, wherein the counter-element is cooled and the belt is heated.

45. A drying device as set forth in claim 42, wherein the belt is heated.

46. A drying device as set forth in claim 45, wherein the counter-element is heated.

47. A drying device as set forth in claim 42, wherein the counter-element comprises a roll configured to be heat conductive with respect to the surface thereof.

48. A drying device as set forth in claim 47, wherein the roll comprises a thermal roll, and wherein an operating temperature of at least one of the thermal roll and the belt is within the range of between about 20° C. and about 400° C.

49. A drying device as set forth in claim 42, wherein the counter-element comprises a fixed support bar configured to be heat conductive with respect to the surface thereof.

50. A device as set forth in claim 42, wherein the belt has a thickness of between about 0.1 mm and about 3 mm and has a tensile strength within the range of between about 10 MPa and about 500 MPa.

51. A device as set forth in claim 42, wherein a thickness of the belt is within the range of between about 0.3 mm and about 1.5 mm.

52. A device as set forth in claim 42, wherein inside the belt is provided at least one press element for pressing the belt against the counter-element.

53. A device as set forth in claim 52, wherein the press element comprises at least one roll selected from the group consisting of a deflection-compensated roll, an elastic surface roll, a polymer-covered roll, a rubber-covered roll, an elastomer surface roll, a shoe roll, a thermal roll, a metal roll, a filled roll, and a composite roll.

54. A device as set forth in claim 42, wherein the press element comprises a second belt loop provided inside the belt.

55. A processing device for drying at least one of a paper web and a board web by pressing, said device comprising at least one of a metal belt and a metal-based belt configured to extend around at least one guiding element, at least one counter-element being disposed outside said belt to establish a contact area with the belt, such that the belt and the counter-element establish therebetween a web processing zone for passing the web to be processed therethrough, wherein the device is provided between the belt and the counter-element with at least one of a porous felt and a wire capable of receiving water to be removed from the fibrous web, wherein a length of the processing zone length is defined by at least one of the disposition of the guiding element and the configuration of said at least one counter-element, wherein a processing time of the web in the processing zone is within the range of between about 10 ms and about 1000 ms, and wherein a contact pressure applied to the web in the processing zone is configured to be within the range of between about 0.01 MPa and about 70 MPa.

56. A device as set forth in claim 55, wherein the belt comprises at least one of a metal belt, a metal wire, a composite metal belt, and a composite metal wire, and wherein the belt is at least partially porous.

57. A device as set forth in claim 56, wherein the counter-element comprises at least one of a second belt loop, a thermal roll, a press roll, a shoe roll, a deflection-compensated roll, an elastic surface roll, a composite roll or some other roll.

58. A device as set forth in claim 57, wherein the counter-element is at least one of configured as a suction roll, configured to have a porous surface, configured to have a perforated surface, and configured to have a grooved surface.

59. A device as set forth in claim 55, wherein the belt is at least one of heated and cooled.

60. A method for drying at least one of a paper web and a board web by pressing in a processing device, comprising a belt configured to extend around at least one guiding element, at least one counter-element being disposed outside said belt to establish a contact area with the belt, such that the belt and the counter-element establish therebetween a web processing zone for passing the web to be processed therethrough, wherein the processing device is provided on both sides of a web with a pore volume, wherein, at least on one side of the web, the pore volume is created in at least one of a compressible felt and wire, wherein the fibrous web to be dried is conveyed in contact with said pore volumes through the processing zone, wherein said volumes are subjected to a compression effect, whereby the at least on of the felt and wire compresses and at the same time the pressure of a gas present in the pores thereof increases, resulting in a gas flow against the web and directing of water present in the web towards the pore volume on the other side of the web.

61. A device as set forth in claim 60, wherein the web is provided on both sides with at least one of a porous felt and wire, each having pore volumes with compressibilities substantially different from each other.

62. A device as set forth in claim 60, wherein the web is provided on one side with at least one of a felt and wire and on the other side with at least one of a porous roll surface and a suction roll.

63. A method for treating at least one of a coated fibrous web and an uncoated fibrous web with a sizing agent, wherein at least one sizing process for the fibrous web to be treated with the sizing agent comprises the use of a processing device as set forth in claim 1 or 18, wherein a contact pressure applied to a web is adjusted by at least one of a tension of the belt and a compression force applied to the belt by press elements optionally provided inside the belt loop to be within the range of between about 0.01 MPa and about 70 MPa.

64. A method as set forth in claim 63, wherein the counter-element comprises a roll selected from the group consisting of a deflection-compensated roll, an elastic surface roll, a polymer-covered roll, a rubber-covered roll, an elastomer surface roll, a shoe roll, a thermal roll, a metal roll, a filled roll, and a composite roll.

65. A method as set forth in claim 63, wherein the counter-element comprises a second belt loop.

66. A method as set forth in claim 63, wherein the method comprises using at least one press element inside the belt for pressing the belt against the counter-element for enhancing a pressure pulse applied to the fibrous web passing through the processing zone.

67. A method as set forth in claim 63, wherein the method comprises at least one of an internal sizing process and a stock sizing process for the fibrous web, wherein the sizing agents are admixed within a stock for producing the web upstream of a headbox of a paper machine, and wherein the fibrous web is then processed in the processing zone for enhancing the sizing process.

68. A method as set forth in claim 67, wherein the fibrous web is processed for a processing time in the processing zone within the range of between about 10 ms and about 1000 ms.

69. A method as set forth in claim 67, wherein the fibrous web is processed for a processing time in the processing zone within the range of between about 50 ms and about 200 ms.

70. A method as set forth in claim 67, wherein a contact pressure applied to the fibrous web passing through the processing zone is adjusted such that the contact pressure applied to the fibrous web in the processing zone is at least equal to a steam pressure generated in processing the fibrous web.

71. A method as set forth in claim 70, wherein the contact pressure is within the range of between about 0.01 MPa and about 70 MPa.

72. A method as set forth in claim 67, wherein a processing temperature of the fibrous web in the processing zone is within the range of between about 100° C. and about 400° C.

73. A method as set forth in claim 67, wherein a temperature of at least one of the counter-element and the belt is raised for processing the fibrous web to a temperature range of between about 100° C. and about 400° C.

74. A method as set forth in claim 73, wherein the temperature is within the range of between about 200° C. and about 300° C.

75. A method as set forth in claim 63, wherein the method comprises using a metal belt calender for surface sizing the fibrous web, and wherein the sizing agents are admixed within the fibrous web on a fibrous-web production line.

76. A method as set forth in claim 75, wherein a processing time of the fibrous web in the processing zone of the metal belt calender is within the range of between about 10 ms and about 300 ms.

77. A method as set forth in claim 76, wherein the processing time of the fibrous web in the processing zone of the metal belt calender is within the range of between about 50 ms and about 200 ms.

78. A method as set forth in claim 75, wherein a processing temperature of the fibrous web in the processing zone of the metal belt calender is within the range of between about 100° C. and about 400° C.

79. A method as set forth in claim 78, wherein the processing temperature is within the range of between about 200° C. and about 300° C.

80. A method as set forth in claim 75, wherein a contact pressure applied to the fibrous web passing through the processing zone is adjusted such that the contact pressure applied to a fibrous web in the processing zone is at least equal to a steam pressure generated in processing the fibrous web.

81. A method as set forth in claim 80, wherein the contact pressure is within the range of between about 0.01 MPa and about 70 MPa.

82. A method as set forth in claim 75, wherein a temperature of at least one of the counter-element and the belt is raised in processing the fibrous web to a temperature range of between about 100° C. and about 400° C.
